(19) United States
(12) Patent Application Publication
(10) Pub. No.: US 2010/0125326 A1
(43) Pub. Date: May 20, 2010

(54) Braided Stent With a Shortenable Tether

(75) Inventors: MEGAN KALSTAD, Santa Rosa, CA (US); YA GUO, Cotati, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/274,862

(22) Filed: Nov. 20, 2008

(51) Int. Cl. A61F 2/86

(52) U.S. Cl. 623/1.11

(57) ABSTRACT

The braided stent with a shortenable tether of the present invention includes a stent for use in a vessel having a vessel wall including a braided stent framework having a first framework end and a second framework end; and a plurality of shortenable tethers, each of the plurality of shortenable tethers having a first tether end and a second tether end, the plurality of shortenable tethers being disposed along a length of the braided stent framework and fixed to the braided stent framework at the first tether end and the second tether end. The plurality of shortenable tethers shorten in response to vessel conditions to urge the first framework end and the second framework end toward each other when the stent is deployed in the vessel to urge a circumference of the braided stent framework toward the vessel wall.

### TECHNICAL FIELD

The technical field of this disclosure is medical implant devices, particularly, braided stents.

### BACKGROUND OF THE INVENTION

Stents are generally cylindrical shaped devices that are radially expandable to hold open a segment of a blood vessel or other anatomical lumen after implantation into the body lumen. Stents have been developed with coatings to deliver drugs or other therapeutic agents.

Stents are used in conjunction with balloon catheters in a variety of medical therapeutic applications including intravascular angioplasty. For example, a balloon catheter device is inflated during PTCA (percutaneous transluminal coronary angioplasty) to dilate a stenotic blood vessel. The stenosis may be the result of a lesion such as a plaque or thrombus. After inflation, the pressurized balloon exerts a compressive force on the lesion thereby increasing the inner diameter of the affected vessel. The increased interior vessel diameter facilitates improved blood flow. Soon after the procedure, however, a significant proportion of treated vessels re-narrow.

To prevent restenosis, short flexible cylinders, or stents, constructed of metal or various polymers are implanted within the vessel to maintain lumen size. The stents acts as a scaffold to support the lumen in an open position. Various configurations of stents include a cylindrical tube defined by a mesh, interconnected stents or like segments. Some exemplary stents are disclosed in U.S. Pat. No. 5,292,331 to Boneau, U.S. Pat. No. 6,090,127 to Globerman, U.S. Pat. No. 5,133,732 to Wiktor, U.S. Pat. No. 4,739,762 to Palmaz and U.S. Pat. No. 5,421,955 to Lau. Balloon-expandable stents are mounted on a collapsed balloon at a diameter smaller than when the stents are deployed. Stents can also be self-expanding, growing to a final diameter when deployed without mechanical assistance from a balloon or like device.

One approach has been to fabricate stents from braided fibers, such as polymer fibers, for making a braided stent with little or no metal. Concern over the long-term effects of stents in the body has led to experimentation with bioabsorbable stents, i.e., stents that are absorbed by the body after deployment. Unfortunately, braided polymer stents often undergo plastic relaxation in the delivery system, leading to a smaller deployment diameter. They also often lack the radial strength to prop open the vessel and maintain a fixed position in the vessel lumen. One approach to alleviate this problem has been to increase the diameter of the fibers forming the braided stent to increase the radial strength. Unfortunately, this increases the crossing profile of the compressed stent, reducing maneuverability and the ability to deploy the stent in smaller vessels. An increased fiber diameter may also increase the time for a bioabsorbable stent to be absorbed and interrupt blood flow dynamics.

Another approach to this problem has been to attach elastomeric axial runners to the braided fiber body. Unfortunately, the elastomeric runners can cause problems during storage and deployment. During storage, the elastomeric runners constantly exert force on the braided fiber body, which can permanently distort the braided fiber body. The elastomeric runners can also lose their elasticity with age and the constant loading and become ineffective. During deployment, the elastomeric runners exert force on the braided fiber body as the braided stent leaves the compressing sheath, so placement of the expanding braided stent is difficult. Due to the high degree of foreshortening of braided stents during deployment, an elastomeric material which has sufficient strength to open a stent would likely deform the undeployed stent when stretched to its full length in the delivery system.

It would be desirable to have a braided stent that would overcome the above disadvantages.

### SUMMARY OF THE INVENTION

One aspect of the present invention provides a stent delivery system including a catheter; and a stent disposed on the catheter. The stent includes a braided stent framework having a first framework end and a second framework end; and a plurality of shortenable tethers, each of the plurality of shortenable tethers having a first tether end and a second tether end, the plurality of shortenable tethers being disposed along a length of the braided stent framework and fixed to the braided stent framework at the first tether end and the second tether end. The plurality of shortenable tethers shorten in response to vessel conditions to urge the first framework end and the second framework end toward each other when the stent is deployed in a vessel to urge a circumference of the braided stent framework toward a vessel wall.

Another aspect of the present invention provides a stent for use in a vessel having a vessel wall including a braided stent framework having a first framework end and a second framework end; and a plurality of shortenable tethers, each of the plurality of shortenable tethers having a first tether end and a second tether end, the plurality of shortenable tethers being disposed along a length of the braided stent framework and fixed to the braided stent framework at the first tether end and the second tether end. The plurality of shortenable tethers shorten in response to vessel conditions to urge the first framework end and the second framework end toward each other when the stent is deployed in the vessel to urge a circumference of the braided stent framework toward the vessel wall.

Another aspect of the present invention provides a stent for use in a vessel having a vessel wall including a braided stent framework having a first framework end and a second framework end; and means for urging the first framework end and the second framework end toward each other in response to vessel conditions when the stent is deployed in the vessel to urge a circumference of the braided stent framework toward the vessel wall.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1 is a perspective view of a stent delivery system made in accordance with the present invention.

FIG. 2 is a side view of a braided stent with a shortenable tether made in accordance with the present invention.

FIGS. 3A-3C are side views of deployment of a braided stent with a shortenable tether made in accordance with the present invention.

FIG. 4 is a side view of another embodiment of a braided stent with a shortenable tether made in accordance with the present invention.

FIG. 5 is a side view of yet another embodiment of a braided stent with a shortenable tether made in accordance with the present invention.

### DETAILED DESCRIPTION OF PRESENTLY PREFERRED EMBODIMENTS

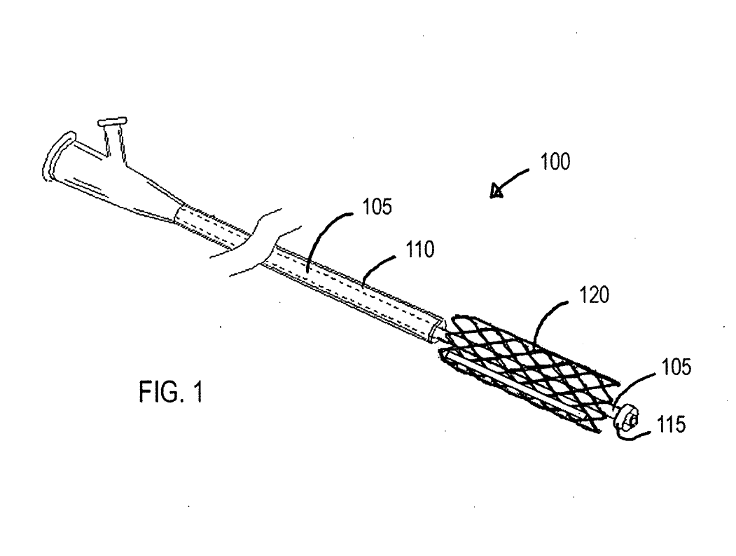

FIG. 1 is a perspective view of a stent delivery system made in accordance with the present invention. In this example, the stent has been advanced from the sheath as it would be for deployment in a vessel. The stent delivery system 100 includes a catheter 105, and a stent 120 disposed on the catheter 105. In one embodiment, a sheath 110 is included in the stent delivery system 100 and the sheath 110 is disposed about the stent 120 to maintain the stent 120 in a compressed state for delivery to the deployment site. In another embodiment, the sheath 110 is omitted and the stent 120 is maintained in the compressed state due to materials or other means of compressing the stent 120. In one embodiment, the catheter 105 can include a retainer 115, such as mechanical or adhesive structures, for retaining the stent 120 on the catheter 105 until the stent 120 is deployed.

The stent 120 can be any variety of braided implantable prosthetic devices known in the art. In one embodiment, the stent 120 can be capable of carrying a coating, such as a polymer coating carrying one or more therapeutic agents, such as anti-inflammatory agents or anti-proliferative agents. In another embodiment, the stent 120 can include one or more therapeutic agents within the stent material. The stent 120 can be bioabsorbable.

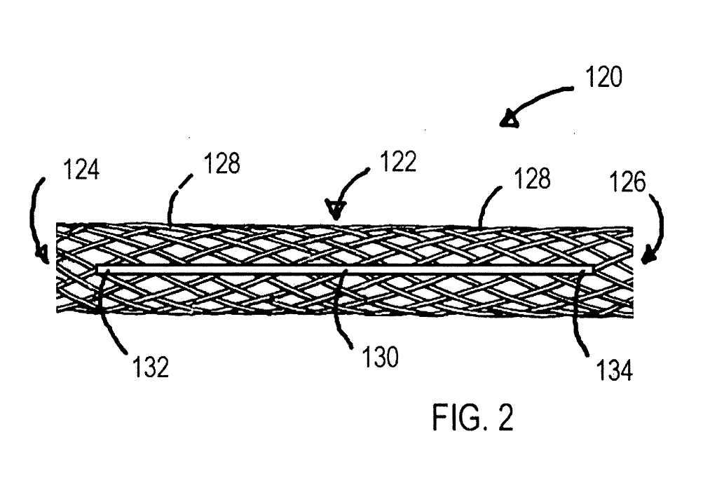

FIG. 2 is a side view of a braided stent with a shortenable tether made in accordance with the present invention. The shortenable tethers are made of a material that shortens in response to vessel conditions to urge the braided stent framework toward each other, thus urging the circumference of the braided stent framework toward the vessel wall. In this embodiment, the shortenable tethers are in a linear pattern, which is defined herein as a pattern in which the shortenable tethers are axially aligned with the length of the stent. The stent 120 can be installed in the stent delivery system of FIG. 1 for implantation in a body lumen.

Referring to FIG. 2, the stent 120 includes a braided stent framework 122 having a first framework end 124 and a second framework end 126, and a number of shortenable tethers 130. In this example, the shortenable tethers 130 are disposed on the outer surface of the braided stent framework 122. Only a single shortenable tether is shown for clarity of illustration. The number, pattern, and location of the shortenable tethers 130 is selected to balance the tension about the circumference of the framework ends when the stent 120 is deployed. For example, when two shortenable tethers are used, the two shortenable tethers can be located 180 degrees apart on the braided stent framework 122. The braided stent framework 122 is formed of a number of fibers 128 braided together to form a generally tubular body. Those skilled in the art will appreciate that the particular braid pattern can be selected as desired for a particular application. In one example, the braid is made up of 16 wires braided in a one over-one under pattern, and the wires are wrapped at a 63 degree angle with respect to the longitudinal axis of a 6 mm mandrel.

The shortenable tethers 130 are shortenable, which is defined herein as having a first length when not deployed in a vessel and having a second length shorter than the first length in response to vessel conditions when deployed in a vessel. The shortenable tethers 130 can shorten in the vessel due to vessel conditions of temperature, exposure to liquid, a combination thereof, or any other vessel condition that causes the material of the shortenable tether 130 to shorten. Each of the shortenable tethers 130 has a first tether end 132 and a second tether end 134. The shortenable tethers 130 are disposed along the length of the braided stent framework 122, and are fixed to the braided stent framework 122 at the first tether end 132 and the second tether end 134. In operation, the shortenable tethers 130 shorten to urge the first framework end 124 and the second framework end 126 toward each other when the stent 120 is deployed in the vessel to urge the circumference of the braided stent framework 122 toward the vessel wall.

The fibers 128 of the braided stent framework 122 are sufficiently flexible and braided in pattern such that urging the framework ends toward each other increases the circumference of the braided stent framework 122. The fibers 128 of the braided stent framework 122 can be made of a wide variety of medical implantable materials, such as stainless steel (particularly 316-L or 316LS stainless steel), MP35 alloy, nitinol, tantalum, ceramic, nickel, titanium, aluminum, degradable and/or nondegradable polymeric materials, tantalum, MP35N, titanium ASTM F63-83 Grade 1, niobium, high carat gold K 19-22, and combinations thereof. The fibers 128 can be single fibers or can be braided. In one example, the fibers 128 can be made of a nondegradable polymer such as polyethylene naphathalate. In another example, the fibers 128 can be made of a bioabsorbable polymer such as poly(lactide-co-glycolide), poly(L-lactide), poly(L,DL,-lactide), poly(lactide-co-lactide-co-trimethylene carbonate), poly(lactide-co-carprolactone), poly(ε-arprolactone), or blends thereof. Those skilled in the art will appreciate that the fibers 128 at the first framework end 124 and second framework end 126 can be free of each other or connected together as desired for a particular application.

The shortenable tethers 130 can be made of any material having a first length when not deployed in a vessel and shortening to a second length shorter than the first length in response to vessel conditions when deployed in a vessel. Exemplary materials include homopolymers and copolymers (including random and block polymers) of D-lactide, L-lactide, DL-lactide, carprolactone, trimethylenecarbonate, glycolide, carprolactone derivatives, P-Dioxanone, and combinations thereof. Polyethylene oxide can be part of the polymer chain. Another exemplary material is degradable polyurethane. In one embodiment, the shortenable tethers 130 can be made of a shape memory polymer that shortens due to temperature change when released from a constraining sheath into physiologic conditions. Exemplary shape memory polymers include block polymers of poly(lactide-b-carprolactone), copolymers of oligo(ε-caprolactone)diol and crystallisable oligo(ρ-dioxanone)diol, and the like. In another embodiment, the shortenable tethers 130 can be made of a shrinkable polymer that shortens when exposed to liquid, heat, or a combination thereof. Exemplary shrinkable polymers include degradable polyurethane, and the like. The shortenable tethers 130 can attain shortenable properties in an initial extrusion or can be subject to a secondary extrusion that softens and draws down the material. The cross section of the shortenable tethers 130 can be circular, rectangular, ellipsoid, or any other cross section as desired for a particular application.

The body of the shortenable tether 130 between the first tether end 132 and the second tether end 134 is sufficiently free to move to be able to urge the first framework end 124 and the second framework end 126 toward each other. In one embodiment, intermediate points on the body of the shortenable tether 130 between the first tether end 132 and the second tether end 134 can also the attached to the inside or the outside of the braided stent framework 122. The shortenable tethers 130 can be attached at the very end of the braided stent framework 122, i.e., at the edge of the first framework end 124 and the second framework end 126, or can be attached a few fiber crossings in from the very end.

Those skilled in the art will appreciate that the shortenable tethers 130 can be attached to the inside or the outside of the braided stent framework 122, i.e., along the vessel wall or within the stent lumen. In another embodiment, the shortenable tethers 130 can be woven through the braided stent framework 122 so that the shortenable tether 130 passes back and forth through the braided stent framework 122.

The number and placement of the shortenable tethers 130 can be selected to balance the tension about the circumference of each of the first framework end 124 and the second framework end 126. In one example, two shortenable tethers 130 can be located 180 degrees apart on the circumference of the braided stent framework 122. In another example, three shortenable tethers 130 can be located 120 degrees apart on the circumference of the braided stent framework 122. In yet another example, a number of shortenable tethers 130 wrap around the braided stent framework 122 in a helical shape to form a crossed or net pattern.

The first tether end 132 and the second tether end 134 can be attached to the braided stent framework 122 with an adhesive or weld. In one example, the adhesive is an ultraviolet curable adhesive. When the stent 120 is bioabsorbable, the adhesive can also be bioabsorbable. Welding processes include heat welding, laser welding, thermal welding, ultrasonic welding, or the like.

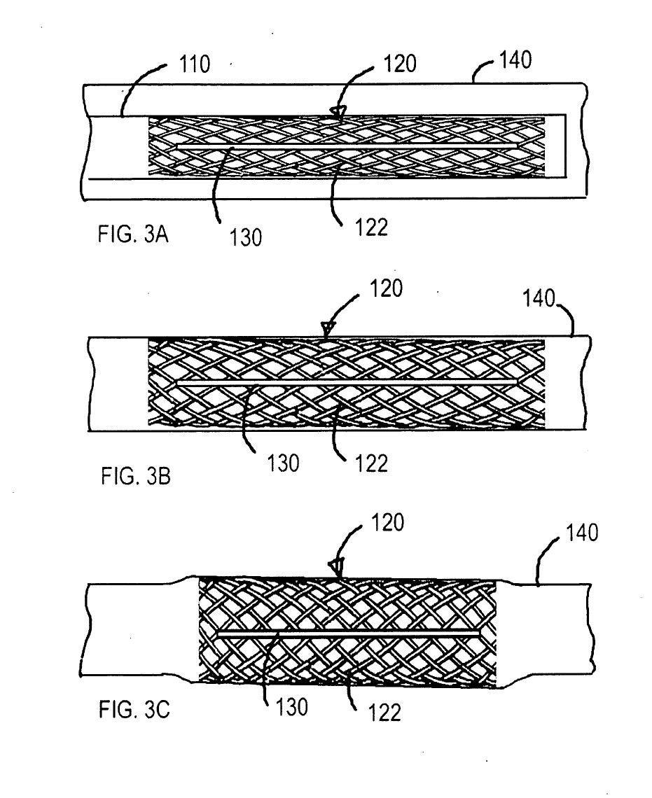

FIGS. 3A-3C are side views of deployment of a braided stent with a shortenable tether made in accordance with the present invention. Referring to FIG. 3A, the stent 120 is advanced through the vessel 140 to the deployment site. The stent 120 is held in a compressed state by the sheath 110. Referring to FIG. 3B, the stent 120 is deployed from the sheath 110 by retracting the sheath 110 or advancing the stent 120 with the catheter (not shown). The braided stent framework 122 expands toward the wall of the vessel 140. The shortenable tether 130 has a first length when the stent 120 exits the sheath 110. Referring to FIG. 3C, the shortenable tether 130 has shortened to a second length, which is shorter than the first length, urging the framework ends toward each other. The circumference of the braided stent framework 122 increases, firmly seating the stent 120 in the vessel 140.

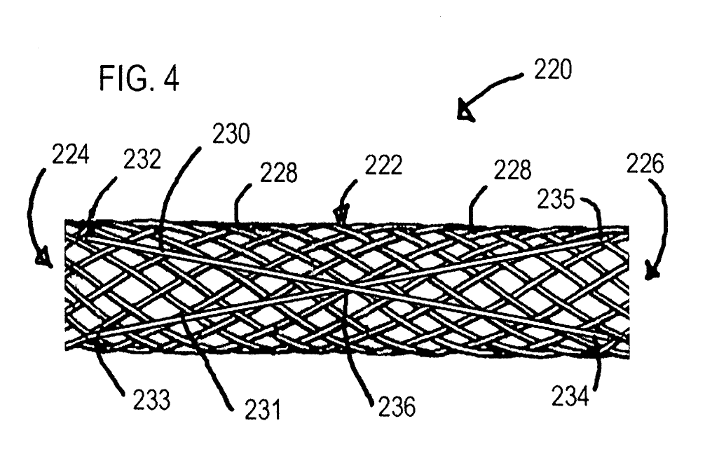

FIG. 4 is a side view of another embodiment of a braided stent with a shortenable tether made in accordance with the present invention. In this embodiment, the shortenable tethers 230 are in a crossed pattern, which is defined herein as a pattern in which the shortenable tethers cross at least once along the length of the stent.

The stent 220 includes a braided stent framework 222 having a first framework end 224 and a second framework end 226, and shortenable tethers 230, 231. In this example, the shortenable tethers 230, 231 wrap around the outside of the braided stent framework 222 in a generally helical arc. Only a single pair of shortenable tethers is shown for clarity of illustration. Typically, another pair of shortenable tethers would be disposed on the opposite side of the braided stent framework 222 to balance the tension about the circumference of framework ends when the stent 220 is deployed. The braided stent framework 222 is formed of a number of fibers 228 braided together to form a generally tubular body.

The shortenable tether 230 has a first tether end 232 and a second tether end 234, and the shortenable tether 231 has a first tether end 233 and a second tether end 235. The shortenable tethers 230, 231 cross at a crossing point 236. The shortenable tethers 230, 231 are disposed along the length of the braided stent framework 222, and are fixed to the braided stent framework 222 at the tether ends. In operation, the shortenable tethers 230, 231 shorten to urge the first framework end 224 and the second framework end 226 toward each other when the stent 220 is deployed in the vessel to urge the circumference of the braided stent framework 222 toward the vessel wall.

In one embodiment, the crossed pattern can be extended into a net pattern encircling the braided stent framework 222. Adjacent tether ends at one framework end can be joined or affixed to the braided stent framework 222 so that the shortenable tethers form a continuous mesh. In one embodiment, a particular shortenable tether can cross more than one other shortenable tether in forming the net pattern, which is defined herein as a crossed pattern that passes around the circumference of the braided stent framework 222. The crossed pattern can be symmetric and at a lower braid angle with respect to the longitudinal axis than fibers making up the braid. Shortenable tethers that arc around the stent framework are subject to less strain during crimping than axial aligned shortenable tethers, and as such, are less likely to plastically deform the stent framework during crimping and storage.

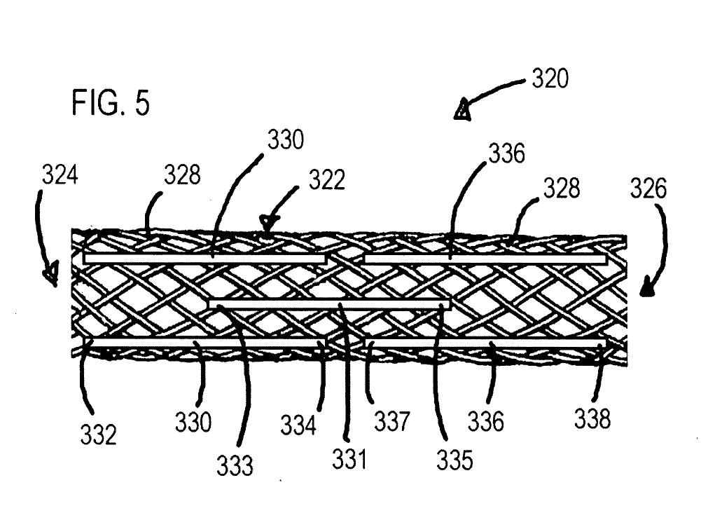

FIG. 5 is a side view of yet another embodiment of a braided stent with a shortenable tether made in accordance with the present invention. In this embodiment, the shortenable tethers are in a staggered pattern, which is defined herein as a pattern in which the shortenable tethers are shorter than the length of the stent and the axial position of at least one shortenable tether overlaps the axial position of another shortenable tether along the length of the stent.

The stent 320 includes a braided stent framework 322 having a first framework end 324 and a second framework end 326, first shortenable tethers 330, second shortenable tethers 331, and third shortenable tethers 336. The braided stent framework 322 is formed of a number of fibers 328 braided together to form a generally tubular body.

The first shortenable tethers 330 have a first tether end 332 and a second tether end 334, the second shortenable tethers 331 have a first tether end 333 and a second tether end 335, and the third shortenable tethers 336 have a first tether end 337 and a second tether end 338. The shortenable tethers 330, 331, 336 are disposed along the length of the braided stent framework 322, and are fixed to the braided stent framework 322 at the tether ends.

The shortenable tethers 330, 331, 336 are staggered, i.e., the axial position of the second tether ends 334 of the first shortenable tethers 330 overlaps the axial position of the first tether ends 333 of the second shortenable tethers 331 and the axial position of the second tether end 335 overlaps the axial position of the first tether ends 337 of the third shortenable tethers 336. In operation, the shortenable tethers 330, 331, 336 shorten to shorten the axial portion of the braided stent framework 322 adjacent the shortenable tethers, urging the circumference of the braided stent framework 322 in that axial portion toward the vessel wall. Those skilled in the art will appreciate that the shortenable tethers can be provided over a single axial portion of the braided stent framework to expand the circumference of just that single axial portion, or can be provided over a number of axial portions of the braided stent framework to expand the circumference of a number of axial portions.

It is important to note that FIGS. 1-5 illustrate specific applications and embodiments of the present invention, and are not intended to limit the scope of the present disclosure or claims to that which is presented therein. Upon reading the specification and reviewing the drawings hereof, it will become immediately obvious to those skilled in the art that myriad other embodiments of the present invention are possible, and that such embodiments are contemplated and fall within the scope of the presently claimed invention.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A stent delivery system comprising:
a catheter; and
a stent disposed on the catheter;
wherein the stent comprises:
a braided stent framework having a first framework end and a second framework end; and
a plurality of shortenable tethers, each of the plurality of shortenable tethers having a first tether end and a second tether end, the plurality of shortenable tethers being disposed along a length of the braided stent framework and fixed to the braided stent framework at the first tether end and the second tether end;
wherein the plurality of shortenable tethers shorten in response to vessel conditions to urge the first framework end and the second framework end toward each other when the stent is deployed in a vessel to urge a circumference of the braided stent framework toward a vessel wall.

2. The stent delivery system of claim 1 further comprising a sheath disposed about the stent.

3. The stent delivery system of claim 1 wherein the braided stent framework and the plurality of shortenable tethers are bioabsorbable.

4. The stent delivery system of claim 1 wherein the plurality of shortenable tethers are disposed along the length of the braided stent framework in a linear pattern.

5. The stent delivery system of claim 1 wherein the plurality of shortenable tethers are disposed along the length of the braided stent framework in a crossed pattern.

6. The stent delivery system of claim 1 wherein the plurality of shortenable tethers are disposed along the length of the braided stent framework in a net pattern.

7. The stent delivery system of claim 1 wherein the plurality of shortenable tethers are disposed along the length of the braided stent framework in a staggered pattern.

8. The stent delivery system of claim 1 wherein the plurality of shortenable tethers are disposed around the braided stent framework in a pattern to balance tension about the circumference of first framework end and the second framework end when the stent is deployed in the vessel.

9. A stent for use in a vessel having a vessel wall comprising:
a braided stent framework having a first framework end and a second framework end; and
a plurality of shortenable tethers, each of the plurality of shortenable tethers having a first tether end and a second tether end, the plurality of shortenable tethers being disposed along a length of the braided stent framework and fixed to the braided stent framework at the first tether end and the second tether end;
wherein the plurality of shortenable tethers shorten in response to vessel conditions to urge the first framework end and the second framework end toward each other when the stent is deployed in the vessel to urge a circumference of the braided stent framework toward the vessel wall.

10. The stent of claim 9 wherein the braided stent framework and the plurality of shortenable tethers are bioabsorbable.

11. The stent of claim 9 wherein the braided stent framework is made of a polymer selected from the group consisting of polyethylene naphathalate, poly(L-lactide), poly(L,DL,-lactide), poly(lactide-co-glycolide), poly(lactide-co-lactide-co-trimethylene carbonate), poly(lactide-co-carprolactone), poly(ε-arprolactone), and blends thereof.

12. The stent of claim 9 wherein the shortenable tethers are made of a polymer selected from the group consisting of block polymers of poly(lactide-b-carprolactone), copolymers of oligo(ε-caprolactone)diol and crystallisable oligo(ρ-dioxanone)diol, and degradable polyurethane.

13. The stent of claim 9 wherein the plurality of shortenable tethers are disposed on an outer surface of the braided stent framework.

14. The stent of claim 9 wherein the plurality of shortenable tethers are disposed along the length of the braided stent framework in a linear pattern.

15. The stent of claim 9 wherein the plurality of shortenable tethers are disposed along the length of the braided stent framework in a crossed pattern.

16. The stent of claim 9 wherein the plurality of shortenable tethers are disposed along the length of the braided stent framework in a net pattern.

17. The stent of claim 9 wherein the plurality of shortenable tethers are disposed along the length of the braided stent framework in a staggered pattern.

18. The stent of claim 9 wherein the plurality of shortenable tethers are disposed around the braided stent framework in a pattern to balance tension about the circumference of first framework end and the second framework end when the stent is deployed in the vessel.

19. The stent of claim 9 wherein the plurality of shortenable tethers are fixed to the braided stent framework by a method selected from the group of adhesive and welding.

20. The stent of claim 9 wherein the vessel conditions are selected from the group consisting of heat, liquid, and combinations thereof.

21. A stent for use in a vessel having a vessel wall comprising:
a braided stent framework having a first framework end and a second framework end; and
means for urging the first framework end and the second framework end toward each other in response to vessel conditions when the stent is deployed in the vessel to urge a circumference of the braided stent framework toward the vessel wall.
